(19) United States
(12) Patent Application Publication
(10) Pub. No.: US 2023/0080356 A1
(43) Pub. Date: Mar. 16, 2023

(54) TELEHEALTH AND MEDICAL IOT COMMUNICATION AND ALERTS

(71) Applicant: BEAMLIVE INC, Woodside, CA (US)

(72) Inventors: Mehrdad Negahban, Woodside, CA (US); Horti Tamás, Budapest (HU); Emilia Tothova, Scunthorpe (GB); Aijaz Hassan, Round Rock, TX (US); Muhammad Usman Khan, Lahore (PK); Ali Akbari, Cupertino, CA (US)

(21) Appl. No.: 17/476,967

(22) Filed: Sep. 16, 2021

(51) Int. Cl. A61B 5/0205; A61B 5/00

(52) U.S. Cl. CPC A61B 5/02055; A61B 5/0004; A61B 5/746; A61B 5/7465

(57) ABSTRACT

This disclosure presents systems and methods to provide telehealth and medical IoT communication. Exemplary implementations may: obtain health information characterizing physiological state of a user; obtain activity information characterizing physical activity of the user; identify potential occurrences of one or more medical-related events based on the health information and the activity information; in response to identifying the potential occurrences of the one or more medical-related events, generate and deliver one or more notifications to one or more computing platforms; and/or perform other features and/or functionality.

### FIELD

This disclosure relates to systems and methods to provide telehealth and medical IoT communication and alerts notifications.

### BACKGROUND

“Telehealth” (sometimes referred to as “telemedicine”) may refer to the distribution of health-related services via electronic information and telecommunication technologies. These technologies may allow long-distance virtual patient and clinician care, monitoring, and/or intervention.

Wearable medical and physical devices have brought medical technology to the hands of consumers. These device may provide a convenient means of monitoring many different physiological features.

### SUMMARY

One or more aspects of the disclosure are related to systems configured to provide telehealth, communication and warnings of medical information through the use of sensors falling within the category of the “Internet-of-Things” (IoT). Wearable devices can connect directly to Internet via WiFi or LTE modems or indirectly to smartphones via Bluetooth. A smartphone app may collect, process, and/or communicate data to a target audience such as doctors or 911 call centers (PSAP). Some wearable devices may be programmed, or a smartphone app may facilitate and provide detection of medical or health related problems and provide warnings to 911 PSAP, doctors, nurses, and/or medical facilities.

The system may be configured to provide live streaming of video and audio, multimedia, readings from Internet of Things (IoT) sensors, and/or other content. The system may be configured to provide an end-to-end platform that facilitates virtual patient/physician visits and provides solutions to the way critical and emergency conditions are detected, alerted, and/or prevented. The system may utilize a HIPAA-compliant cloud which securely connects medical devices and/or sensors (such as smart watches and/or other wearable sensors that include heartbeat sensor, blood pressure sensor, temperature sensor, sugar level sensor, oxygen level sensor, respiratory rate sensor, and/or other sensors), to smartphones and browsers. The system may utilize a patient-sanctioned, always-on connection via a software API to a medical device and facilitates output via an analytics or artificial intelligence (AI) tool.

In some implementations, the system may be configured to collect vital signs (heath information) such as heartbeat, blood pressure, temperature, sugar level, oxygen level, respiratory rate and/or other physiological data from wearable medical devices and:

1) Overlay that with video (and/or audio) and GPS using a sub-second latency connectivity for next generation of telehealth;

2) Compare Vital signs (health information) to Physical activity (activity information) such as Sleeping, seating, walking, running, exercising, etc., to generate Real time Alerts such as but not limited to: Green—need to see a doctor; Amber—must see a doctor now; Red—medical emergency; and

3) Generate Analytics that compares the user data (health and physical information) with a database of large number of existing users to provide prior information required for machine learning or Al to primarily achieve objectives of item 2.

One or more implementations of the system and methods present herein may integrate details of vital signs with physical activity status of a person in real-time. This integration may allow physicians to accurately gauge a patient's dynamic activity-specific vital signs, including their reaction to various stages of movement and/or exertion. The system may achieve this by overlaying real-time vital signs and/or physical activity from medical sensors/devices with a camera to create a new level of virtual visits—regardless of patient and physician geographical locations. The real-time observation of a patient's physical activity may be extracted from GPS, accelerometer, and/or other sensors. The system may enable one or more of real-time virtual visits which provide effective remote testing and prognosis, medical alerts based on automatic and/or instant detection of abnormalities or pre-set statistical emergency alerts, medical analytics that identifies preventive analytics to improve health and standard of living, and/or other features and/or functionality that may become apparent to a person of ordinary skill in the art.

One aspect of the present disclosure relates to a system configured for telehealth and medical IoT communication and alerts. The system may include one or more hardware processors configured by machine-readable instructions and/or other components. The machine-readable instructions may include one or more computer program components. The computer program components may include one or more of a health component, an activity component, an analytics component, a page component, a notification component, a meeting component, and/or other computer program components.

The health component may be configured to obtain health information derived from output signals generated by one or more sensors. The health information may characterize physiological state of a user. The physiological state may comprise a measure of one or more of blood pressure, heart rate, body temperature, blood oxygen level, respiratory rate, blood sugar level, and/or other measure of physiological state.

The activity component may be configured to obtain activity information characterizing physical activity of the user. The physical activity of the user may comprise active and/or passive activity. The physical activity may be characterized as one or more of sleeping, seating, walking, running, exercising, falling, and/or other activity.

The analytics component may be configured to identify potential occurrences of one or more medical-related events based on one or more of the health information, the activity information, and/or other information.

The notification component may be configured to, in response to identifying the potential occurrences of the one or more medical-related events, generate and deliver one or more notifications to one or more computing platforms.

The page component may be configured to generate, by a cloud-based page service, a dynamic health page for the user. The health information and/or the activity information may be stored in the dynamic health page in an ongoing manner as the health information and the activity information are obtained. A page may facilitate real time telehealth connectivity using video (and/or audio) overlaid by Medical IoT sensors that communicates, in sub-second latency, vital and physical activities of a patient to a physician to observe.

The meeting component may be configured to effectuate presentation of a meeting interface through which a user conducts a shared virtual meeting with a medical professional. The meeting interface may include a portion of a page. The meeting interface may be configured to display one or more of the health information, the activity information, and/or other information obtained in real time during the shared virtual meeting.

As used herein, any association (or relation, or reflection, or indication, or correspondence) involving servers, processors, computing platforms, and/or another entity or object that interacts with any part of the system and/or plays a part in the operation of the system, may be a one-to-one association, a one-to-many association, a many-to-one association, and/or a many-to-many association or N-to-M association (note that N and M may be different numbers greater than 1).

As used herein, the term “obtain” (and derivatives thereof) may include active and/or passive retrieval, determination, derivation, transfer, upload, download, submission, and/or exchange of information, and/or any combination thereof. As used herein, the term “effectuate” (and derivatives thereof) may include active and/or passive causation of any effect, both local and remote. As used herein, the term “determine” (and derivatives thereof) may include measure, calculate, compute, estimate, approximate, generate, and/or otherwise derive, and/or any combination thereof.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 illustrates a system configured to provide telehealth and medical IoT communication and alerts, in accordance with one or more implementations.

FIG. 2 illustrates a method to provide telehealth and medical IoT communication and alerts, in accordance with one or more implementations.

FIG. 3 illustrates a user interface displaying a baseline health profile, in accordance with one or more implementations.

FIG. 4 illustrates a user interface showing presentation of health information and/or activity information, in accordance with one or more implementations.

FIG. 5 illustrates a user interface displaying a dynamic health page, in accordance with one or more implementations.

FIG. 6 illustrates a meeting interface, in accordance with one or more implementations.

### DETAILED DESCRIPTION

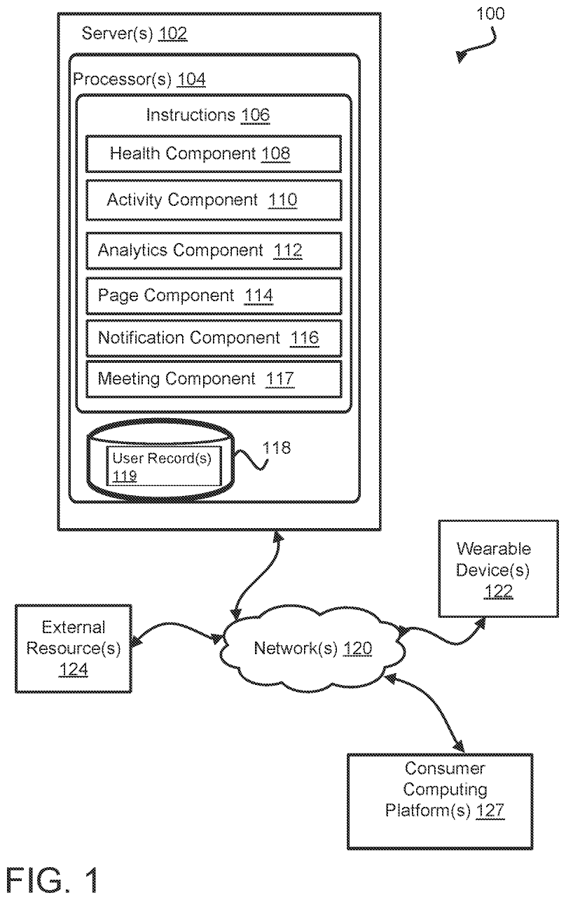

FIG. 1 illustrates a system 100 configured to provide telehealth and medical IoT communication and alerts, in accordance with one or more implementations. The system 100 may be configured to integrate details of vital signs with physical activity of a person in real-time. This integration may allow users and/or physicians to accurately gauge dynamic vital signs and/or reaction to various stages of movement and/or exertion. The system 100 may achieve this by overlaying real-time vital signs from medical sensors/devices with a camera to create a new level of virtual visits—regardless of patient and physician geographical locations. The real-time observation of a patient's physical activity may be extracted from one or more of GPS, accelerometer, and/or other sensors. The system 100 may enable one or more of real-time virtual visits which provide effective remote testing and prognosis, medical alerts based on automatic and/or instant detection of abnormalities or pre-set statistical emergency alerts, medical analytics that identifies preventive analytics to improve health and standard of living, and/or other features and/or functionality that may become apparent to a person of ordinary skill in the art.

The system 100 may include one or more of one or more servers 102, one or more wearable devices 122, one or more consumer computing platforms 127, one or more external resources 124, and/or other components.

An individual wearable device of one or more wearable devices 122 may comprise one or more of a medical device, a consumer electronic device, and/or other devices. An individual wearable device may be configured to be worn, held, and/or otherwise engaged to a user. In some implementations, an individual wearable device may be a device that falls within a category of the Internet of things (IoT). The Internet of things (IoT) describes, generally, a network of physical objects embedded with sensor(s), software, and/or other technologies for the purpose of connecting and/or exchanging information with other devices and/or systems over the Internet and/or other networks.

In some implementations, an individual wearable device may actively and/or passively intervene with a subject. “Active” intervention may refer to invasive engagement based on one or more of an implantation and/or an insertion of a device or portion thereof into the body of the subject. “Passive” intervention may refer to noninvasive contact with the body of the subject and/or an as-used disposition at or near the body (e.g., skin) of the subject. In some implementations, an individual wearable device may have a form factor of one or more of watch, a necklace, a waist band, a chest band, an implant, a facemask, a medical device configured for one or more specific medical purposes, and/or other forms.

An individual wearable device may include one or more of non-transitory electronic storage, one or more sensors, one or more transceivers, and/or other components. The one or more transceivers may be configured for wireless communication. The wireless communication may include Bluetooth, Wi-Fi, Near-filed communication, and/or other wireless communication protocol. The one or more transceivers may be configured to transmit and/or receive radio-frequency (RF) signals. The transmitted and/or received signals may convey information.

Individual sensors of a wearable device may be configured to generate output signals conveying information usable by system 100. The one or more sensors may include one or more of an orientation sensor, a location sensor, a pressure sensor, a temperature sensor, a blood oxygen sensor, a heartrate sensor, a blood pressure sensor, a respiration sensor, an audio input sensor, blood sugar sensors, and/or other sensors.

In some implementations, an orientation sensor may be configured to generate output signals conveying orientation information and/or other information. Orientation information derived from output signals of an orientation sensor may define an orientation of an individual wearable device. In some implementations, orientation may refer to one or more of a pitch angle, a roll angle, a yaw angle, heading, pointing direction, and/or other measurements. An orientation sensor may include an inertial measurement unit (IMU) such as one or more of an accelerometer, a gyroscope, a magnetometer, Inclinometers, Electronic nose, Infrared Imagers, Micro-bolometers, micro-displays (DMD), Digital micro-mirror device, Optical Switches, and/or other devices.

In some implementations, a location sensor may be configured to generate output signals conveying location information and/or other information. Location information derived from output signals of a location sensor may define one or more of a geo-location of an individual wearable device, an elevation of an individual wearable device, and/or other measurements. A location sensor may include one or more of a GPS, an altimeter, and/or other devices.

A pressure sensor may be configured to generate output signals conveying pressure information and/or other information. Pressure information derived from output signals of a pressure sensor may define a force per unit area imparted to the pressure sensor. A pressure sensor may include one or more of a strain-gauge, a piezo resistive strain gauge, a capacitive pressure sensor, an electromagnetic pressure sensor, a piezoelectric sensor, and/or other pressure sensors.

A temperature sensor may be configured to generate output signals conveying temperature information and/or other information. Temperature information derived from output signals of a temperature sensor may define one or more of a temperature at the temperature sensor, temperature within a threshold range of the temperature sensor, temperature of an object in contact with the temperature sensor (e.g., body temperature of a user), and/or other measure of temperature. A temperature sensor may include one or more of a thermocouple, a resistive temperature Measuring devices, an infrared sensor, a bimetallic device, a thermometer, and/or other temperature sensors.

A blood oxygen sensor may be configured to generate output signals conveying blood oxygen information and/or other information. The blood oxygen information may include oxygen saturation level (e.g., oxygen levels in blood) and/or other information. The blood oxygen sensor may be configured to detect changes in how efficiently oxygen is being carried. The blood oxygen sensor may include one or more of a light source, a light sensor, and/or components. A light source may be configured to emit light so that light pass through a user's skin. The light sensor may be configured to measure the amount of light that reflects back. The light reflecting back on the light sensor may convey changes of light absorption in oxygenated and/or deoxygenated blood. A blood oxygen sensor may comprise, for example, a pulse oximeter.

A heartrate sensor may be configured to generate output signals conveying heartrate information and/or other information. The heartrate information may include a heart rate of a subject. The heartrate sensor may include one or more of a light source, a light sensor, and/or other components. The light source may be configured to emit light so that light pass through a user's skin. The light sensor may be configured to measure the amount of light that reflects back. The light reflections will vary as blood pulses under your skin past the light. The variations in the light reflections may be interpreted as heartbeat.

A blood pressure sensor may be configured to generate output signals conveying blood pressure information and/or other information. The blood pressure information may include a measure of blood pressure. The measure may be one or more of blood pressure at a specific point in time, an average blood pressure over a period of time, and/or other measures of blood pressure. A blood pressure sensor may include one or more of a radar sensor, passive infrared (PIR) sensor, ECG, photoplethysmography (PPG) sensor, and/or other device.

A respiration sensor may be configured to generate output signals conveying respiration information and/or other information. The respiration information may include one or more of breathing rate, volume of breadth, and/or other information. A respiration sensor may include one or more of a piezoresistive sensor (to be placed over the chest with a strap), a pressure sensor (that take advantage of the events of diaphragm contraction and relaxation, an acoustic sensor (to monitor lung sounds), an accelerometer (to capture respiratory movements during inhalation and exhalation), and/or other device.

An audio input sensor may be configured to receive audio input. An audio input sensor may include a sound transducer and/or other sensor configured to convert sound (e.g., air pressure variation) into an electrical signal. By way of non-limiting illustration, an audio input sensor may include a microphone.

A blood sugar sensor may be configured to generate output signals conveying blood sugar information and/or other information. The blood sugar information may include a level of blood sugar in a user's body, and/or other information.

Individual consumer computing platforms of one or more consumer computing platforms 127 may include devices through which consumers access system 100. An individual consumer computing platform may include one or more of a cellular telephone, a smartphone, a laptop, a tablet computer, a virtual reality platform, a game console, a camera, a desktop computer, a mixed reality headset, augmented reality device, and/or other consumer computing platform. In some implementations, an individual consumer computing platform may include one or more of one or more processors, a display, one or more sensors, and/or other components. A display may be configured to present visual content. The visual content may include one or more of individual images, image sequences, video, text, and/or other content. Presentation via a display of a consumer computing platform may be facilitated by one or more processors of the consumer computing platform and/or other components. By way of non-limiting example, presentation may be facilitated by a graphics processing unit (GPU) and/or other components. A display may include one or more of a screen, a touchscreen, a monitor, a head-mounted display, a set of screens, and/or other displays. The one or more sensors of an individual computing platform may include one or more of an orientation sensor, a location sensor, a pressure sensor, a temperature sensor, an audio input sensor, one or more other sensors described herein, and/or other sensors.

In some implementations, output signals generated by one or more sensors of one or more wearable devices 122 and/or one or more consumer computing platforms 127 may include and/or convey one or more of health information, activity information, and/or other information. The health information may characterize physiological state of a user. The health information may include measures commonly referred to as “vital signs.” The physiological state may comprise a measure of one or more of blood pressure, heart rate, body temperature, oxygen level, respiratory rate, and/or other measure of physiological state which may be derived from output signals of one or more sensor. The activity information may characterize physical activity of the user. The physical activity of the user may comprise active and/or passive activity. The physical activity may be characterized as one or more of sleeping, seating, walking, running, falling, and/or other activity.

The one or more servers 102 may include one or more of one or more physical processors 104, non-transitory electronic storage 118, and/or other components. The non-transitory electronic storage 118 may be configured to store information utilized by one or more servers 102, one or more wearable devices 122, one or more consumer computing platforms 127, and/or other components of system 100.

In some implementations, one or more servers 102 may be configured to provide remote hosting of features and/or functions of machine-readable instructions 106 to one or more consumer computing platforms 127 and/or other components. The one or more wearable devices 122, one or more consumer computing platforms 127, and/or other components may be remotely located from one or more servers 102. The one or more servers 102 may communicate with the one or more wearable devices 122, one or more consumer computing platforms 127, and/or other components via client/server architecture, and/or other communication schemes. In some implementations, individual consumer computing platforms may download an application (e.g., non-transitory electronically stored information) configured to provide access to features and/or functions of machine-readable instructions 106. In some implementations, output signals generated by one or more sensors of one or more wearable devices may be routed to server(s) 102 via individual computing platforms.

In some implementations, one or more features and/or functions of one or more servers 102 may be attributed as local features and/or functions of individual ones of the one or more consumer computing platforms 127. For example, individual consumer computing platforms may obtain and/or include machine-readable instructions that may be the same or similar to machine-readable instructions 106 of one or more physical processors 104.

In FIG. 1, one or more physical processors 104 of server(s) 102 may be configured by machine-readable instructions 106. Executing machine-readable instructions 106 may cause one or more physical processors 104 to facilitate telehealth and medical IoT communication and alerts. The machine-readable instructions 106 may include one or more computer program components. The one or more computer program components may include one or more of a health component 108, an activity component 110, an analytics component 112, a page component 114, a notification component 116, a meeting component 117, and/or other components.

The health component 108 may be configured to obtain health information and/or other information. The health component 108 may be configured to obtain health information and/or other information from one or more wearable devices 122 and/or one or more consumer computing platforms 127. The health component 108 may be configured to obtain output signals generated by one or more sensors of one or more wearable devices 122 and/or one or more consumer computing platforms 127. The health component 108 may be configured to determine and/or derive the health information from the output signals.

The activity component 110 may be configured to obtain activity information and/or other information. The activity component 110 may be configured to obtain activity information and/or other information from one or more wearable devices 122. The activity component 110 may be configured to obtain output signals generated by one or more sensors of one or more wearable devices 122 and/or one or more consumer computing platforms 127. The activity component 110 may be configured to determine and/or derive the activity information from the output signals. By way of non-limiting illustration, based on location sensor output signals (and/or other output signals), activity component 110 may be configured to determine whether a user is walking, running, laying down, exercising, sleeping, and/or performing other activity.

In some implementations, the activity component 110 may utilize machine learning and/or other techniques to determine physical activity from output signals of one or more sensors. In some implementations, a machine learning model may be trained on a training data set comprising input/output pairs. An input may include output signals of one or more sensors. An output may comprise an indication of the activity being performed. In some implementations, a user may help to train the model by performing specific activities (according to instructions given to them) while the activity component 110 obtains output signals.

The analytics component 112 may be configured to generate one or more user records 119. The one or more user records 119 may be stored in electronic storage 118 and/or other storage locations. The one or more user records 119 may be generated based on information determined by analytics component 112 and/or obtained by one or more of health component 108, activity component 110, and/or other components.

An individual user record may be associated with an individual user. In a given user record associated with a user, the health information for the user may be stored in association with the activity information for the user. The information may be stored in an ongoing manner as the information is obtained. The health information may be stored in association with the activity information so that health information at a given point in time (or over a period of time) may be correlated with activity information at the given point in time (or over the period of time). The correlation may overlay the health information with the activity information to provide context to the health information. For example, if a relatively high heart rate is measured, but correlated with activity such as running, then there may no need for concern. However, if a relatively high heart rate is measured, and correlated with activity such as sleeping, then there may be need for further action.

The analytics component 112 may be configured to generate baseline health profiles for individual users based on the information stored in the user records. Baseline health profiles may be an establishment of healthy and/or otherwise “normal” vitals of individual users in relation to different forms of physical activity. From the baseline health profiles, the analytics component 112 may be configured to identify potential occurrences of one or more medical-related events. A medical-related event may correspond to a dangerous and/or abnormal physiological state in relation to physical activity that may warrant a medical emergency visit. The baseline health profiles may include measures of one or more of blood pressure, heartbeat rate, body temperature, blood oxygen level, respiratory rate, and/or other information.

A relatively normal body temperature may range from 97.8 degrees Fahrenheit (36.5 degrees Celsius) to 99 degrees F. (37.2 degrees C.) for a healthy adult.

A relatively normal resting heart rate for adults ranges from 60 to 100 beats a minute. Generally, a lower resting heart rate may imply more efficient heart function and better cardiovascular fitness. Although there can be a wide range of “normal”, an unusually high or low heart rate may indicate an underlying problem.

Blood pressure may comprise a measure of the force of the blood pushing against the artery walls. Relatively high blood pressure directly increases the risk of coronary heart disease (heart attack) and stroke (brain attack). With high blood pressure, the arteries may have an increased resistance against the flow of blood, causing the heart to pump harder to circulate the blood.

The respiration rate (breathing rate) may be measured by the number of breaths a person takes per minute and/or other unit of time. Relatively normal respiration rates for a resting adult range from 12 to 16 breaths per minute. Respiration rates may increase with fever, illness, and/or with other medical conditions. In many cases, changes in respiratory rate may be the first indication of a possible deterioration, much sooner than changes in other vital signs.

Oxygen saturation (or blood oxygen levels), abbreviated as SpO2, may comprise a measure of the fraction of oxygen saturated-hemoglobin relative to the total hemoglobin in the blood. Relatively normal blood oxygen levels in humans may be considered 95-100 percent. Any level lower than 95% may compromise organ function, so consistent monitoring of this vital sign may be crucial.

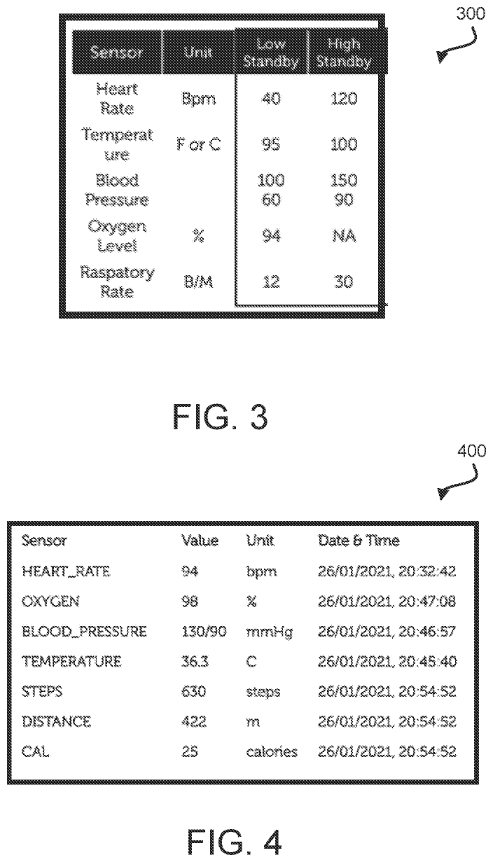

As mentioned above, baseline health profiles may include the health information stored in association with the activity information for the user. Accordingly, in some implementations, a user may be associated with a set of multiple baseline health profiles, wherein an individual baseline health profile corresponds to an individual physical activity. Typical human physical activity states may include one or more of sleeping, sitting, standing, walking, running, collision/collapsing/falling, and/or other activity. FIG. 3 illustrates a user interface 300 displaying a baseline health profile, in accordance with one or more implementations. In the user interface 300, the baseline health profile may be shown with respect to a “low” and “high” range. However, the baseline health profile may be expressed in other ways. The user interface 300 may show the baseline health profile via grid of columns, rows, and cells.

Returning to FIG. 1, in some implementations, the analytics component 112 may be configured to identify potential occurrences of one or more medical-related events based on one or more of the health information, the activity information, the baseline health profiles, and/or other information. In some implementations, the analytics component 112 may be configured to perform a comparison between the health information and the activity information as it is currently being obtained with the health information and the activity information previously obtained and stored in the user record (e.g., the baseline health profiles). In some implementations, performing the comparison may include one or more of determining a current physical activity of a user based on the activity information, obtaining a baseline health profile corresponding to the current physical activity, comparing the health information and the activity information as it is currently being obtained with the obtained baseline health profile, and/or other operations. In some implementations, comparing may include determining difference between the current information and the baseline health profiles.

The analytics component 112 may be configured to identify the potential occurrences of the one or more medical-related events based on the comparison and/or other information. In some implementations, potential occurrence of one or more medical-related events may be identified when the difference between the health information and the activity information as it is currently being obtained and the baseline health profile(s) reaches or exceeds a threshold difference. In some implementations, a threshold may be 1 to 10 percent of the measured baseline health profile data. In some implementations, a threshold may be more than 10 percent of the measured baseline health profile data. In some implementations, a threshold may be specified by a user, a physician, and/or other entity. By way of non-limiting illustration, an average baseline heartrate while running may be measured to be 160 beats per minute. If a current heartrate while running is measured at 170 beats per minute, this may not trigger the analytics component 112 to identify a potential occurrences of a medical-related event (less than 10% increase over baseline). On the other hand, if a current heartrate while running is measured at 220 beats per minute, this may trigger the analytics component 112 to identify a potential occurrences of a medical-related event (more than 10% increase over baseline).

In some implementations, analytics component 112 may be configured to identify the potential occurrences of the one or more medical-related events based on machine learning and/or other techniques. In some implementations, a machine learning model may be trained on a training data set comprising input/output pairs. An input may include one or more of health information, activity information, and/or other information. An output may comprise an indication of a medical-related event and/or other information. In some implementations, the machine learning model may be trained based on available health data, medical literature, and/or other content. In some implementations, a training data set may include the information the user records of one or more users of system 100. The user records may act as a database having a large number of existing users to provide prior information required for machine learning or AI to primarily achieve the objective of identifying potential occurrences of one or more medical-related events.

The notification component 116 may be configured to, in response to identifying potential occurrences of one or more medical-related events, generate and/or deliver one or more notifications to one or more computing platforms and/or other resources. A computing platform receiving a notification may be a computing platform of a user themselves, a computing platform of another user or individual listed as an emergency contact, a computing platform of a resource, and/or other computing platform. In some implementations, an individual notification may include one or more of the health information, activity information, one or more alerts, and/or other information. Real time alerts may include, but are not limited to: Green—need to see a doctor, Amber—must see a doctor now, Red—medical emergency, and/or other alerts.

In some implementations, an individual notification may include a prompt that a user may need medical assistance. In some implementations, an individual notification may prompt a user if they are “ok” and/or if they need emergency services. In some implementations, an individual notification may include a request for support services from a resource. The request may include a location of a user where help should be sent. In some implementations, a resource may include one or more of a public safety answering point (PSAP), police, firefighters, ambulance services, and/or other resource. A notification may be delivered over the network(s) 120 and/or other communication channel (e.g., SMS). In some implementations, a medical professional may obtain a notification of alert which they may use to report certain events and/or warnings to medical team or 911 PSAP.

FIG. 4 illustrates a user interface 400 presenting health information and/or activity information, in accordance with one or more implementations. The user interface 400 may include live (e.g., real-time) information and/or information at a given snapshot of time.

Returning to FIG. 1, the analytics component 112 may be configured to identify actual occurrences of the one or more medical-related events based on one or more of the health information, the activity information, the baseline health profile(s), and/or other information. In some implementations, actual occurrences of the one or more medical-related events may be identified when user vitals exhibit measures that are indicative of actual medical problems where emergency intervention may be required.

The notification component 116 may be configured to, in response to identifying the actual occurrences of the one or more medical-related events, generate and/or deliver one or more communications to one or more resources. A communication may include a request for support services from a resource. A communication sent in response to identifying actual occurrences may include the same as or similar content as a notification sent in response to identifying the potential occurrences.

The meeting component 117 may be configured to effectuate presentation of a meeting interface through which a user conducts shared virtual meetings with medical professionals. In some implementations, a meeting interface may be provided by a meeting resource. In some implementations, a meeting interface may be provided as part of a page. In some implementations, a meeting resource may be configured to implement an instance of a meeting through one or more meeting interfaces. A meeting resource may be configured to receive and/or transmit the communications (e.g., textual communications, voice communications, video communications, etc.) that make up a meeting to and/or from consumer computing platform(s) 127 and server(s) 102. A meeting interface may be part of and/or external to one or more user interfaces provided by the system 100 itself. A meeting interface may be hosted by the system 100 and/or one or more third party applications integrated with system 100 via an application program interface (API). A meeting interface may include its own interface and/or a portion a user interface provided by the system 100.

In some implementations, a meeting interface may be presented in response to a specific request by a user to conduct a meeting and/or another user to accept the meeting request. In some implementations, the meeting interface may be presented in response to occurrence of a date and/or time of the meeting.

The meeting interface may include one or more portions. The meeting interface may include a health display portion displaying one or more of health information, activity information, other information obtained in real time during the shared virtual meeting, user records (e.g., baseline health profiles), and/or other information. The meeting interface may include a video conference portion showing live video feeds and presenting live audio feeds of the participants in a shared virtual meeting. The health display portion may be a set size, in a set position in the meeting interface, or the user may customize the size and/or positioning of the health display portion. The meeting interface may include a notification portion to display notifications.

By way of non-limiting illustration, a meeting interface may be presented on a first computing platform associated a user, second computing platform associated with a medical professional, and/or other computing platforms. The meeting interface as presented on the second computing platform may include a health display portion displaying the health information and the activity information. This may allow the medical professional to assess real time health data. In some implementations, the second computing platform may receive one or more notifications as described with respect to notification component 116.

By way of non-limiting illustration, a patient's virtual visit may allow medical professionals to view vital signs (e.g., health information) and physical observation in tandem. Vital signs may be overlaid with live video/audio and physical activity (e.g., activity information) enabling more effective remote testing and prognosis.

By way of non-limiting illustration, in the virtual visit for example, a physician can ask a patient wearing a medical sensor/device to stand, walk, and then run (in-place or on a treadmill). Based on resulting heartbeat rates and oxygen levels and observation, the physician can identify possible need for additional testing, including for Covid, COPD, AFib, and/or other considerations.

FIG. 6 illustrates a meeting interface 600, in accordance with one or more implementations. The meeting interface 600 may include one or more portions. The meeting interface 600 may include one or more of a health display portion 604, a video conference portion 602, a notification portion 606, and/or other portions. The health display portion 604 may be configured to display one or more of health information, activity information, and/or other information obtained in real time during the shared virtual meeting. The video conference portion 602 may show live video feeds of the participants in a shared virtual meeting. Live audio may be presented. The notification portion 606 may be configured to display notifications.

Returning to FIG. 1, the page component 114 may be configured to generate dynamic health pages for the users. A dynamic health page (also referred to herein simply as “a page”) may include a web page and/or destination where information may be dynamically stored, accessed, updated, communicated, and/or interacted with in other ways. The individual pages may be accessed via links (e.g., embedded URLs), and/or other types of access. Individual pages may be stored in electronic storage 118 and/or other storage locations. The individual pages may be associated with addresses (e.g., network addresses) where assets defining the individual pages are located within electronic storage 118.

The page component 114 may act as by a cloud-based page service through which the dynamic health pages of the users are created, modified, updated, and/or shared. A page may include an integrated set of content. The content of a page may be updated dynamically and/or continuously as information is generated. In some implementations, the content of a dynamic health page for a given user may include a user record for the user, live update of health information and/or activity information, and/or other information. In some implementations, a page may include an instance of a meeting interface through which users and physicians conduct virtual meetings (see, e.g., meeting component 117). In some implementations, the use of a page to provide a meeting interface may overlay video with both the health information and the activity information in sub-second latency.

The page component 114 may be configured to generate page identifiers for the dynamic health pages. An individual page identifier may identify (e.g., point to) an individual dynamic health page. An individual page identifier may provide access to an individual page. In some implementations, the page component 114, acting as a cloud-based page service, may be configured to generate and/or deliver notification(s) the same as or similar to notification component 116. In some implementations, notifications generated and/or delivered by page component 114 may include a page identifier for a corresponding page.

In some implementations, individual pages may include scrollable user interfaces. A page may include a vertical arrangement of content which may be scrolled through to view different portions of the content. Scrolling may be accomplished through user input, for example, finger swiping a touchscreen display of a computing platform.

The page component 114 may be configured to receive output signals generated by the one or more sensors and include the output signals in the dynamic health pages. The page component 114 may be configured to determine the health information from the output signals of one or more sensors and include the health information in the dynamic health pages. The page component 114 may be configured to determine the activity information from the output signals of one or more sensors and include the activity information in the dynamic health pages.

In some implementations, the page component 114 may receive the output signals directly from one or more sensors wirelessly over network(s) 120. In some implementations, the page component 114 may receive the output signals from one or more sensors routed through one or more consumer computing platforms 127.

The page component 114 may be configured to effectuate presentation of a user interface on individual consumer computing platforms of one or more consumer computing platforms 127. Effectuating presentation may include sending control signals and/or other signals and/or commands to the individual consumer computing platforms to cause the individual consumer computing platforms to present a user interface. An instance of the user interface may be configured to display a dynamic health page.

In some implementations, one or more users may have access to a page associated with a subject. By way of non-limiting illustration, medical staff and/or other users may have access to a page associated with one of their patients. In some implementations, one or more users may be configured to modify and/or control the content of a page. In some implementations, modifying and/or controlling the content of a page may include controlling one or more sensors that provide output readings that populate information on a page. The page component 114 may be configured to receive user input from a user (e.g., medical personnel) identifying which sensor(s) worn by a patient should be reporting information to a page. By way of non-limiting illustration, page component 114 may be configured to obtain, from a second computing platform associated with a medical professional that is remote from a patient, input information conveying user input identifying individual ones of sensors that should contribute to the determinations of health information and/or activity information presented in the interface. Accordingly, the system 100 can allow a medical staff to remotely decide which sensor to report live via the cloud page or directly to them.

The page component 114 may be configured to receive user input from a user (e.g., medical professional) including adjustments to the thresholds and/or other information which dictate the reporting of certain events, alerts, and/or warnings to a medical team or 911 PSAP. In some implementations, a dedicated computing platform application may be installed on the computing platforms of medical professionals which allow them to provide input remotely from the patient. In some implementations, a medical professional, such as a doctor, may set one or more thresholds which will allow them to qualify a call to 911 PSAP or allow emergency response to be aware of the medical or physical conditions of a subject.

In some implementations, page component 114 may be configured to retrieve a template that serves as a basis for creating a page. The template may be configured by a user in order to customize a page that is created based on the template. The template may include default information to include (or exclude). In some implementations, templates may have at least some information or elements that are common to each template. In some implementations, different templates may be created and used for different communication channels. For example, a page that is to be shared via a broadband communication channel (e.g., high-speed Internet) may be formatted differently than a page that is to be shared via Short Message Service (“SMS”) text. For example, a page shared over SMS text may include less detail and content than a page shared over the Internet. In some implementations, page component 114 may be configured to generate different versions of the page based on the communication channel over which the page is to be communicated. In some implementations, the communication channel over which the page is to be communicated may be determined based on the type, configuration, and/or capabilities of the individual consumer computing platform which is accessing the page.

In some implementations, page component 114 may identify a recipient computing platform that should receive a page based on input from another user, a time, a location of the recipient, a user profile for the recipient, and/or other information used to identify recipients. In some implementations, page component 114 may identify a recipient of a page based on user input. For example, page component 114 may receive an identification of a user with whom the page should be shared. The identification may be received from the user who requested to access the page and/or other users. In some implementations, page component 114 may identify one or more communication channels over which the page is capable of being communicated based on the recipient computing platform.

In some implementations, the page component 114 may be configured to monitor user engagement with the individual pages. User engagement may include one or more of time-spent viewing a page, amount and/or direction of scrolling, and/or other information. The page component 114 may be configured to effectuate changes to the individual pages in response to the user engagement. Changes may include one or more of adding content to the individual pages that was previously not on the pages, removing content from the individual pages, replacing content of the individual pages with other content that was previously not on the pages, and/or other changes.

In some implementations, the page component 114 may be configured to create and/or manage workflows by providing integrated workflow tools with the pages. The page component 114 may allow users (e.g., physicians, guardians, etc.) to view and/or update health status of a user (e.g., patients, relatives, etc.). In some embodiments, page component 114 may provide various workflow tools. A user may interface with the workflow tools to determine to whom, and/or under what conditions, individual notifications and/or other communication should be sent. The workflow tools may be used to specify other actions to be carried out in response to currently obtained health information and/or activity information and/or in response to identification of potential and/or actual occurrences of medical related events.

In some implementations, the page component 114 may be configured to send out a communication/notification independent from a page (e.g., via an email to a contact's email address, a text message to a phone number, a call to a phone number, and/or other communication) in response to changes health information and/or activity information. In some implementations, page component 114 may be configured to notify various users with whom the page has been shared of the creation, update, expiration, and/or other status updates related to the page.

In some implementations, page component 114 may cause a notification that a shared page has been updated (e.g., new baseline established) to be provided to one or more recipients. A messaging service (e.g., SMS text message, email, social media platform, etc.) may be used to convey the notifications/communications.

In some implementations, a communication independent from a page may include instructions to carry out one or more actions. The communication including instructions to carry out one or more actions may be communicated to a user and/or another computer system. A communication including instructions to carry out one or more actions may be sent to a computer system associated with an emergency response resource. By way of non-limiting illustration, an identification of a potential and/or actual occurrences of the one or more medical-related events may cause a communication to be sent to a resource (e.g., PSAP, firefighters, etc.) which requests an ambulance to a location of a user experience the medical emergency. Other actions are contemplated.

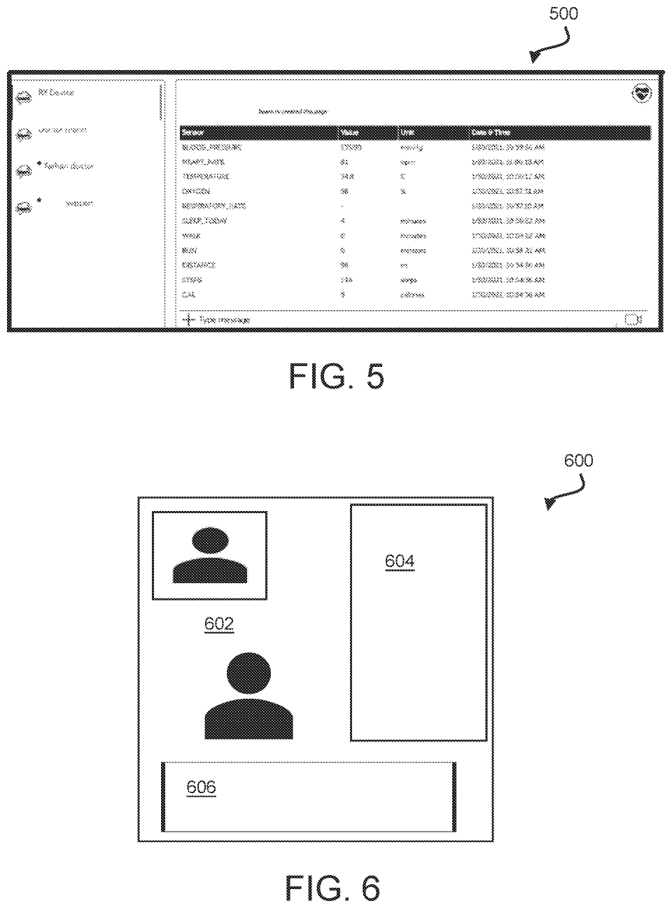

FIG. 5 illustrates a user interface 500 displaying a dynamic health page, in accordance with one or more implementations. The user interface 500 may be presented on a consumer computing platform. The dynamic health page may include an integrated set of content, which may include one or more of health information, activity information, baseline health profiles, notifications, and/or other information.

It is noted that the examples shown and described with respect to FIG. 3-FIG. 6 are for illustrative purposes only and not to be considered limiting. Instead, those skilled in the art may appreciate other configurations of user interfaces that convey the information described herein.

Returning to FIG. 1, server(s) 102, one or more wearable devices 122, one or more consumer computing platforms 127, external resource(s) 124, and/or other components may be operatively linked via one or more electronic communication links. For example, such electronic communication links may be established, at least in part, via one or more networks 120. One or more networks 120 may include the Internet, Bluetooth, Near field communication, and/or other networks. It will be appreciated that this is not intended to be limiting and that the scope of this disclosure includes implementations in which components of system 100 may be operatively linked via some other communication media.

External resource(s) 124 may include sources of information, hosts, and/or providers of information outside of system 100, external entities participating with system 100, and/or other resources. In some implementations, some or all of the functionality attributed herein to external resource(s) 124 may be provided by resources included in system 100.

Server(s) 102 may include communication lines or ports to enable the exchange of information with one or more of a network, wearable device(s) 122, consumer computing platform(s) 127, and/or other components. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 118 may include electronic storage media that electronically stores information. The electronic storage media of electronic storage 118 may include one or both of system storage that is provided integrally (i.e., substantially non-removable) with server(s) 102 and/or removable storage that is removably connectable to server(s) 102 via, for example, a port or a drive. A port may include a USB port, a firewire port, and/or other port. A drive may include a disk drive and/or other drive. Electronic storage 118 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. The electronic storage 118 may include one or more virtual storage resources (e.g., cloud storage, a virtual private network, and/or other virtual storage resources). Electronic storage 118 may store software algorithms, information determined by processor(s) 104, information received from consumer computing platform(s) 127, and/or other information that enables system 100 to function as described herein.

Processor(s) 104 may be configured to provide information-processing capabilities in server(s) 102. As such, processor(s) 104 may include one or more of a digital processor, an analog processor, a digital circuit designed to process information, an analog circuit designed to process information, a state machine, and/or other mechanisms for electronically processing information. Although processor(s) 104 is shown in FIG. 1 as a single entity, this is for illustrative purposes only. In some implementations, processor(s) 104 may include one or more processing units. These processing units may be physically located within the same device, or processor(s) 104 may represent processing functionality of a plurality of devices operating in coordination. The processor(s) 104 may be configured to execute components 108, 110, 112, 114, 116, and/or 117. Processor(s) 104 may be configured to execute components 108, 110, 112, 114, 116, and/or 117 by software; hardware; firmware; some combination of software, hardware, and/or firmware; and/or other mechanisms for configuring processing capabilities on processor(s) 104.

It should be appreciated that although components 108, 110, 112, 114, 116, and/or 117 are illustrated in FIG. 1 as being co-located within a single processing unit, in implementations in which processor(s) 104 includes multiple processing units, one or more of components 108, 110, 112, 114, 116, and/or 117 may be located remotely from the other components. While computer program components are described herein as being implemented via processor(s) 104 through machine readable instructions 106, this is merely for ease of reference and is not meant to be limiting. In some implementations, one or more functions of computer program components described herein may be implemented via hardware (e.g., dedicated chip, field-programmable gate array). One or more functions of computer program components described herein may be one or more of software-implemented, hardware-implemented, and/or software and hardware-implemented. The description of the functionality provided by the different components 108, 110, 112, 114, 116, and/or 117 described above is for illustrative purposes and is not intended to be limiting, as any of components 108, 110, 112, 114, 116, and/or 117 may provide more or less functionality than is described. For example, one or more of components 108, 110, 112, 114, 116, and/or 117 may be eliminated, and some or all of its functionality may be provided by other ones of components 108, 110, 112, 114, 116, 117 and/or other components. As another example, processor(s) 104 may be configured to execute one or more additional components that may perform some or all of the functionality attributed to one of components 108, 110, 112, 114, 116, and/or 117.

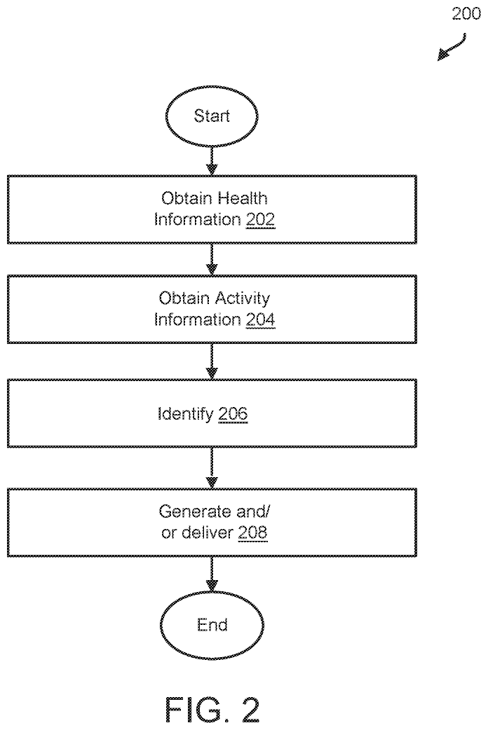

FIG. 2 illustrates a method 200 to provide telehealth and medical IoT communication and alerts. The operations of method 200 presented below are intended to be illustrative. In some implementations, method 200 may be accomplished with one or more additional operations not described, and/or without one or more of the operations discussed. Additionally, the order in which the operations of method 200 are illustrated in FIG. 2 and described below is not intended to be limiting.

In some implementations, method 200 may be implemented in a system comprising one or more processing devices (e.g., a digital processor, physical processor, an analog processor, a digital circuit designed to process information, an analog circuit designed to process information, a state machine, and/or other mechanisms for electronically processing information), storage media storing machine-readable instructions, one or more wearable devices, one or more consumer computing platforms, and/or other components. The one or more processing devices may include one or more devices executing some or all of the operations of method 200 in response to instructions stored electronically on electronic storage media. The one or more processing devices may include one or more devices configured through hardware, firmware, and/or software to be specifically designed for execution of one or more of the operations of method 200.

At an operation 202, health information may be obtained. The health information may be derived from output signals generated by one or more sensors. The health information may characterize physiological state of a user. In some implementations, operation 202 may be performed by one or more physical processors executing a component the same as or similar to health component 108 (shown in FIG. 1 and described herein).

At an operation 204, activity information characterizing physical activity of the user may be obtained. In some implementations, operation 204 may be performed by one or more physical processors executing a component the same as or similar to activity component 110 (shown in FIG. 1 and described herein).

At an operation 206, potential occurrences of one or more medical-related events may be identified based on one or more of the health information, the activity information, and/or other information. In some implementations, operation 206 may be performed by one or more physical processors executing a component the same as or similar to analytics component 112 (shown in FIG. 1 and described herein).

At an operation 208, in response to identifying the potential occurrences of the one or more medical-related events, one or more notifications may be generated and/or delivered to one or more computing platforms. In some implementations, operation 208 may be performed by one or more physical processors executing a component the same as or similar to notification component 116 (shown in FIG. 1 and described herein).

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

What is claimed is:

1. A system configured for telehealth and medical IoT communication and alerts, the system comprising:
one or more physical processors configured by machine-readable instructions to:
obtain health information derived from output signals generated by one or more sensors, the health information characterizing physiological state of a user;
obtain activity information characterizing physical activity of the user;
identify potential occurrences of one or more medical-related events based on the health information and the activity information; and
in response to identifying the potential occurrences of the one or more medical-related events, generate and deliver one or more notifications to one or more computing platforms.

2. The system of claim 1, wherein the one or more physical processors are further configured by the machine-readable instructions to:
store, in a user record associated with the user, the health information in association with the activity information in an ongoing manner as the health information and the activity information are obtained;
perform a comparison between the health information and the activity information as it is currently being obtained with the health information and the activity information previously obtained and stored in the user record; and
identify the potential occurrences of the one or more medical-related events based on the comparison.

3. The system of claim 2, wherein the one or more physical processors are further configured by the machine-readable instructions to:
generate, by a cloud-based page service, a dynamic health page for the user, wherein the user record is stored in the dynamic health page.

4. The system of claim 3, wherein the one or more physical processors are further configured by the machine-readable instructions to:
generate, by the cloud-based page service, a page identifier for the dynamic health page; and
wherein individual notifications in the one or more notifications include the page identifier.

5. The system of claim 1, wherein the physiological state is a measure of one or more of blood pressure, heart rate, body temperature, oxygen level, blood sugar level, or respiratory rate; and
wherein the physical activity of the user is characterized as one or more of sleeping, seating, walking, running, exercising, or falling.

6. The system of claim 1, wherein the one or more physical processors are further configured by the machine-readable instructions to:
identify actual occurrences of the one or more medical-related events based on the health information and the activity information; and
in response to identifying the actual occurrences of the one or more medical-related events, generate and deliver one or more communications including alerts and notifications in real time to one or more medical emergency response resources.

7. The system of claim 1, wherein the one or more physical processors are further configured by the machine-readable instructions to:
receive, at a cloud-based page service, the output signals generated by the one or more sensors;
determine, at the cloud-based page service, the health information from the output signals;
generate, at the cloud-based page service, the one or more notifications; and
deliver the one or more notification from the cloud-based page service to the one or more computing platforms.

8. The system of claim 7, wherein the one or more physical processors are further configured by the machine-readable instructions to:
receive, at the cloud-based page service, second output signals generated by one or more second sensors coupled to a computing platform associated with the user;
determine, at the cloud-based page service, the activity information from the second output signals.

9. The system of claim 8, wherein the cloud-based page service receives the output signals directly from the one or more sensors wirelessly over a network.

10. The system of claim 1, wherein the one or more physical processors are further configured by the machine-readable instructions to:
effectuate presentation of a meeting interface through which the user conducts a shared virtual meeting with a medical professional, such that the meeting interface is presented on a first computing platform associated the user and a second computing platform associated with the medical professional;
wherein the meeting interface as presented on the second computing platform includes a health display portion displaying the health information and the activity information; and
wherein the one or more computing platforms that are delivered the one or more notifications includes the second computing platform.

11. A method to provide telehealth and medical IoT communication, the method comprising:
obtaining health information derived from output signals generated by one or more sensors, the health information characterizing physiological state of a user;
obtaining activity information characterizing physical activity of the user;
identifying potential occurrences of one or more medical-related events based on the health information and the activity information; and
in response to identifying the potential occurrences of the one or more medical-related events, generating and delivering one or more notifications to one or more computing platforms.

12. The method of claim 11, further comprising:
storing, in a user record associated with the user, the health information in association with the activity information in an ongoing manner as the health information and the activity information are obtained;
performing a comparison between the health information and the activity information as it is currently being obtained with the health information and the activity information previously obtained and stored in the user record; and
identifying the potential occurrences of the one or more medical-related events based on the comparison.

13. The method of claim 12, further comprising:
generating, by a cloud-based page service, a dynamic health page for the user, wherein the user record is stored in the dynamic health page.

14. The method of claim 13, further comprising:
generating, by the cloud-based page service, a page identifier for the dynamic health page; and
wherein individual notifications in the one or more notifications include the page identifier.

15. The method of claim 11, wherein the physiological state is a measure of one or more of blood pressure, heart rate, body temperature, oxygen level, blood sugar level, or respiratory rate; and
wherein the physical activity of the user is characterized as one or more of sleeping, seating, walking, running, exercising, or falling.

16. The method of claim 11, further comprising:
identifying actual occurrences of the one or more medical-related events based on the health information and the activity information; and
in response to identifying the actual occurrences of the one or more medical-related events, generating and delivering one or more communications including alerts and notifications in real time to one or more medical emergency response resources.

17. The method of claim 11, further comprising:
receiving, at a cloud-based page service, the output signals generated by the one or more sensors;
determining, at the cloud-based page service, the health information from the output signals;
generating, at the cloud-based page service, the one or more notifications; and
delivering the one or more notification from the cloud-based page service to the one or more computing platforms.

18. The method of claim 17, further comprising:
receiving, at the cloud-based page service, second output signals generated by one or more second sensors coupled to a computing platform associated with the user; and
determining, at the cloud-based page service, the activity information from the second output signals.

19. The method of claim 18, wherein the cloud-based page service receives the output signals directly from the one or more sensors wirelessly over a network.

20. The method of claim 11, further comprising:
effectuating presentation of a meeting interface through which the user conducts a shared virtual meeting with a medical professional, including presenting the meeting interface on a first computing platform associated the user and a second computing platform associated with the medical professional;
wherein the meeting interface as presented on the second computing platform includes a health display portion displaying the health information and the activity information; and
wherein the one or more computing platforms that are delivered the one or more notifications includes the second computing platform.

21. The system of claim 8, wherein the one or more physical processors are further configured by the machine-readable instructions to:
obtain, from a second computing platform associated with a medical professional that is remote from the computing platform, input information conveying user input by the medical professional identifying individual ones of the one or more sensors and the one or more second sensors that contribute to determinations of the health information and the activity information.

22. The system of claim 10, wherein the one or more notifications are delivered to the second computing platform based on thresholds specified by the medical professional.
